(19) United States
(12) Patent Application Publication
(10) Pub. No.: US 2020/0390389 A1
(43) Pub. Date: Dec. 17, 2020

(54) METHOD AND SYSTEM FOR MOTOR FUNCTION REHABILITATION AND MONITORING A PATIENT'S RECOVERY

(71) Applicant: NEC Corporation, Minato-ku, Tokyo (JP)

(72) Inventors: Ni Ni SOE, BENDEMEER ROAD (SG); Charles CHOY, BENDEMEER ROAD (SG); Elaine GOMEZ, SIMEI STREET (SG); Fiona Min Fang TAN, SIMEI STREET (SG)

(73) Assignee: NEC Corporation, Minato-ku, Tokyo (JP)

(21) Appl. No.: 16/957,245

(22) PCT Filed: Jan. 25, 2019
(86) PCT No.: PCT/JP2019/002523

§ 371 Date: Jun. 23, 2020

(30) Foreign Application Priority Data
Feb. 5, 2018 (SG) 1020180097IR

(51) Int. Cl. A61B 5/00; A61B 5/0488; A61B 5/11; A63B 71/06; G16H 40/67; G16H 20/30; G16H 50/30; G16H 50/70; G09B 19/00

(52) U.S. Cl. CPC A61B 5/4848; A61B 5/0488; A61B 5/1124; A63B 71/0622; G16H 40/67; G16H 20/30; A63B 2071/0625; G16H 50/70; G09B 19/003; A61B 2505/09; A61B 2562/0219; A61B 2562/06; G16H 50/30

(57) ABSTRACT

A method and system for monitoring and determining progress of a patient's rehabilitative treatment are provided. The system includes a patient output device, a plurality of sensory devices and a processing means. The patient output device displays visual and/or presents auditory instructions to the patient. The plurality of sensory devices sense physiological performance and body portion movement while the patient moves two or more portions of the patient's body in response to the visual and/or auditory instructions. The processing means generates first data in response to the sensed movement of a first portion of the patient's body, generates second data in response to the sensed movement of a second portion of the patient's body, and determines an objective functional recovery level representing the patient's rehabilitative treatment progress in response to all of a manifested category, a latent category and a significant category of the first data and the second data.

### TECHNICAL FIELD

The present invention generally relates to methods, devices and systems for patient functional ability determination, and more particularly relates to methods, devices and systems for motor function rehabilitation and monitoring a patient's recovery.

### BACKGROUND ART

In today's world, brain injuries, including brain injuries resulting from stroke, are a leading cause of death and a leading increase in a patients' post-injury disability. The ability to live independently after a brain injury depends largely on the patient's recovery of motor function and functional abilities after the brain injury. Therefore, accurate assessment of functional abilities provides substantial assistance for rehabilitation planning and support realistic goal-setting by clinicians, therapists and patients him/herself.

In addition, it is important to understand a patient's current functional condition and progress in rehabilitation treatment in order to provide suitable treatment strategies. To understand the current stage of a patient's functional abilities, quantitative determination of motor function is a strong clinically relevant indicator of treatment effectiveness. However, current rehabilitation processes are based on subjective scoring of a functional assessment of selected patient movements by a trained clinical therapist.

In addition, in remote monitoring rehabilitation systems, most assessments use motion-sensing to assess motor function to quantify the functional abilities, yet motion-sensing rarely captures the actual functional performance of a patient. If only motion-sensing is used, important information of muscle activity is not captured and relevant functional performance of a patient is not reflected in scoring manual muscle testing (MMT). Further, existing solutions are not suitable for early stage functional assessment of a patients' recovery, such as functional assessment within the first seventy-two hours in an acute hospital environment.

Thus, what is needed is an objective assessment of motor function and quantification of functional abilities which reflects the individual patient's functional abilities whether obtained in person in early stage recovery or obtained later by telemonitoring a patient's recovery. Furthermore, other desirable features and characteristics will become apparent from the subsequent detailed description and the appended claims, taken in conjunction with the accompanying drawings and this background of the disclosure.

### SUMMARY OF INVENTION

According to at least one embodiment of the present invention, a method for monitoring and determining progress of a patient's rehabilitative treatment is provided. The method includes sensing physiological performance and body portion movement while moving two or more portions of the patient's body in response to visual and/or auditory instructions, generating first data in response to the sensed movement of a first portion of the patient's body, and generating second data in response to the sensed movement of a second portion of the patient's body. The method further includes determining an objective functional recovery level representing the patient's rehabilitative treatment progress in response to all of a manifested category, a latent category and a significant category of the first data and the second data.

According to another embodiment of the present invention, a system for monitoring and determining progress of a patient's rehabilitative treatment is provided. The system includes a patient output device, a plurality of sensory devices and a processing means. The patient output device displays visual and/or presents auditory instructions to the patient. The plurality of sensory devices sense physiological performance and body portion movement while the patient moves two or more portions of the patient's body in response to the visual and/or auditory instructions. The processing means generates first data in response to the sensed movement of a first portion of the patient's body, generates second data in response to the sensed movement of a second portion of the patient's body, and determines an objective functional recovery level representing the patient's rehabilitative treatment progress in response to all of a manifested category, a latent category and a significant category of the first data and the second data.

According to another embodiment of the present invention, a non-transitory computer readable medium containing program instructions for causing a computer to perform a method for monitoring and determining progress of a patient's rehabilitative treatment is provided. The method includes receiving first physiological performance data in response to a sensed physiological performance of a first portion of the patient's body, receiving first sensed movement data in response to a sensed movement of the first portion of the patient's body, and segmenting the first physiological performance data and the first sensed movement data to generate first data. The method also includes receiving second physiological performance data in response to a sensed physiological performance of a second portion of the patient's body, receiving second sensed movement data in response to a sensed movement of the second portion of the patient's body, and segmenting the second physiological performance data and the second sensed movement data to generate second data. Finally, the method includes determining an objective functional recovery level representing the patient's rehabilitative treatment progress in response to all of a manifested category, a latent category and a significant category of corresponding segmented portions of the first data and the second data.

### BRIEF DESCRIPTION OF DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with a present embodiment.

FIG. 1 depicts an illustration of a system for monitoring and determining progress of a patient's rehabilitative treatment in accordance with a present embodiment.

FIG. 2 depicts a flowchart of operation of a system for monitoring and determining progress of a patient's rehabilitative treatment in accordance with the present embodiment.

FIG. 3 depicts an illustration of the system for monitoring and determining progress of a patient's rehabilitative treatment of FIG. 1 in accordance with the present embodiment.

FIG. 4 depicts a table of parameters of the system for monitoring and determining progress of a patient's rehabilitative treatment of FIG. 1 in accordance with the present embodiment.

FIG. 5 depicts a sample of a visualization feedback system of FIG. 1 in accordance with the present embodiment.

FIG. 6 depicts a flowchart illustrating a method for patient data collection for determining progress of a patient's rehabilitative treatment in accordance with the present embodiment.

FIG. 7 depicts a flowchart illustrating a method for patient test trials for data collection in accordance with the flowchart of FIG. 6 for determining progress of a patient's rehabilitative treatment in accordance with the present embodiment.

FIG. 8 depicts a flowchart illustrating sensor signal processing in accordance with the present embodiment.

FIG. 9 depicts a flowchart illustrating maximum volume contraction of electromyography (EMG) data extraction in accordance with the present embodiment.

FIG. 10 depicts a block diagram of the processing of the system of FIG. 1 for monitoring and determining progress of a patient's rehabilitative treatment in accordance with the present embodiment.

FIG. 11 depicts a block diagram of the objective score determination in accordance with the present embodiment.

FIG. 12 depicts a block diagram of recovery level index determination in accordance with the present embodiment.

FIG. 13 depicts a more detailed block diagram of recovery level index determination of FIG. 12 in accordance with the present embodiment.

FIG. 14 depicts a flowchart of clustering in accordance with the present embodiment.

FIG. 15 depicts a flowchart for determining recovery level in response to the clustering of FIG. 14 in accordance with the present embodiment.

Skilled artisans will appreciate that elements in the figures are illustrated for simplicity and clarity and have not necessarily been depicted to scale.

### DESCRIPTION OF EMBODIMENTS

The following detailed description is merely exemplary in nature and is not intended to limit the invention or the application and uses of the invention. Furthermore, there is no intention to be bound by any theory presented in the preceding background of the invention or the following detailed description. It is the intent of the present embodiment to present a fully automated functional assessment method and system for monitoring and determining progress of a patient's rehabilitative treatment which uses a combination of electromyography (EMG) and inertial measurement unit (IMU) signals to quantify the performance of the patient's functional abilities. In this manner, present embodiments provide a fully automated motor functional assessment system for stroke rehabilitation and monitoring a patient's stroke recovery which covers minimum requirements of early stage functional assessment. A system is provided which quantitatively defines a performance-based recovery level by providing a subjective score and recovery level. A minimal and noninvasive early prognosis system for stroke recovery is provided which is able to assess the patients' condition along the recovery journey including at early stages of recovery such as within seventy-hours after stroke onset and/or hospital admission. Monitoring patient's functional recovery level in accordance with present embodiments will enable early and continuous planning treatment strategies. In addition, the systems and methods in accordance with present embodiments provides an objective patient functional recovery level of upper or lower limb which can be fully interpretable by a doctor, a therapist and even by the patient him/herself.

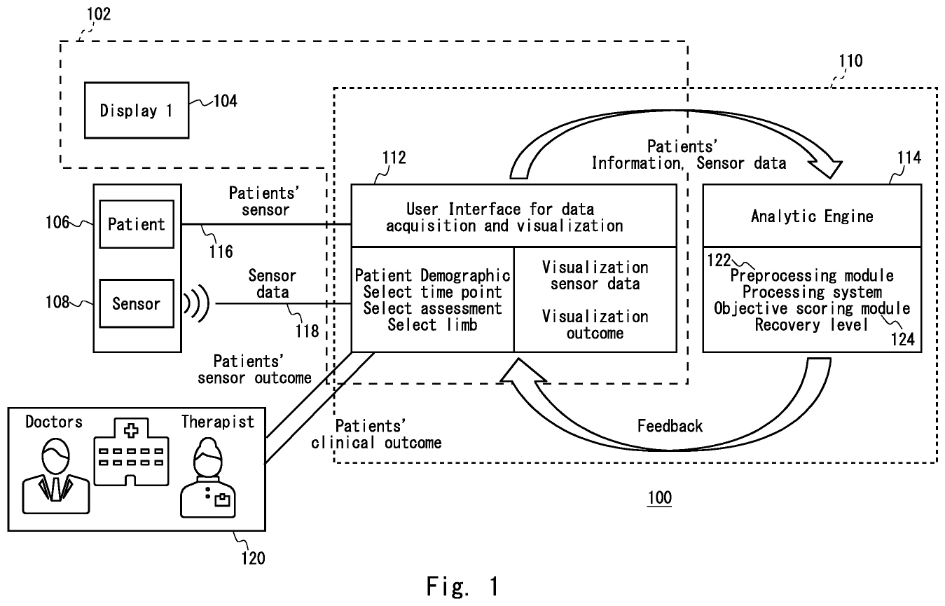

Referring to FIG. 1, an illustration 100 of a system for monitoring and determining progress of a patient's rehabilitative treatment in accordance with a present embodiment. The system includes a patient output device 102, such as a display 104 for displays visual instructions to a patient 106. Instead of visual instructions, in accordance with the present embodiment an output device can be provided to present auditory instructions to the patient 106.

The system also includes a plurality of sensory devices 108 and a processing means 110 including a user interface 112 and an analytic engine 114. As the patient output device 102 displays visual and/or presents auditory instructions to the patient, the plurality of sensor devices 108 are activated by a signal 116 and sensor data 118 is obtained. The user interface 112 presents visualization of the sensor data for the patient 106 as well as for professionals 120, such as doctors, therapists and other clinicians or hospital personnel. The professionals 120 can provide additional information, data or instructions to the processing means 110.

In accordance with the present embodiment, the plurality of sensory devices 108 sense physiological performance and body portion movement while the patient 106 moves two or more portions of the patient's body in response to the visual and/or auditory instructions. The processing means 110 generates first data in response to the sensed movement of a first portion of the patient's body and generates second data in response to the sensed movement of a second portion of the patient's body. In accordance with the present embodiment, the analytic engine includes a preprocessing module 122, and a processing system which includes an objective scoring module 124 which determines an objective functional recovery level representing the patient's rehabilitative treatment progress in response to all of a manifested category, a latent category and a significant category of the first data and the second data.

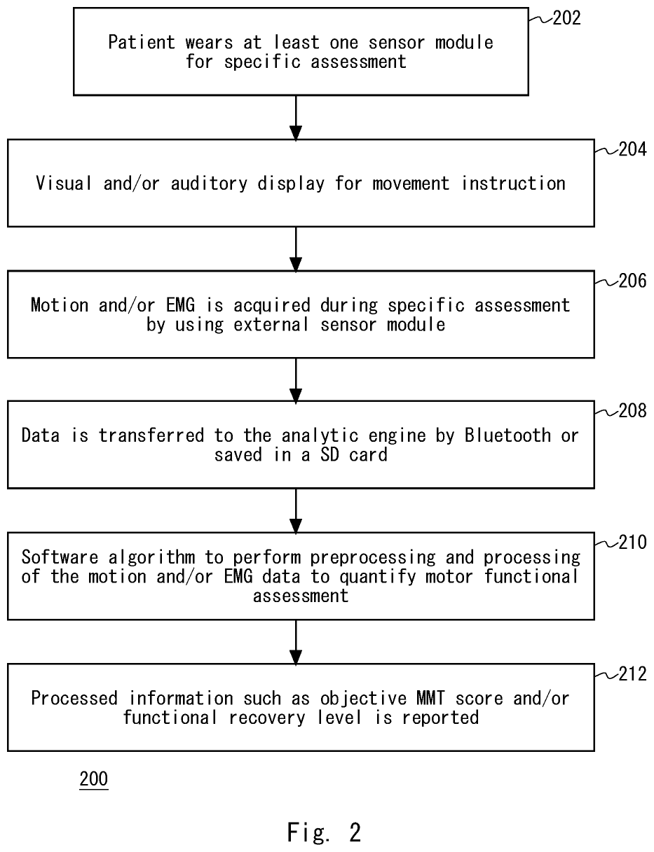

Referring to FIG. 2, a flowchart 200 depicts operation of the system for monitoring and determining progress of the patient's rehabilitative treatment in accordance with the present embodiment. At step 202, the patient 106 wears at least one sensor module 108 for specific assessment of functional abilities. At step 204, the display 104 and/or the user interface 112 presents visual and/or auditory movement instructions. At step 206, range of motion and/or electromyography (EMG) signals are acquired to sense physiological performance (e.g., EMG) and body portion movement while moving the patient 106 moves two or more portions of the patient's body in response to the visual and/or auditory instructions.

At step 208, first and second data is generated and transferred 118 to the analytic engine 114, the first data generated in response to the sensed movement of a first portion of the patient's body and the second data generated in response to the sensed movement of a second portion of the patient's body. At step 210, processing of the motion and/or EMG data determines an objective functional recovery level representing the patient's rehabilitative treatment progress in response to all of a manifested category, a latent category and a significant category of the first data and the second data. And at step 212, the objective functional recovery level is reported to the patient 106 via the user interface 112 and to the professionals 120.

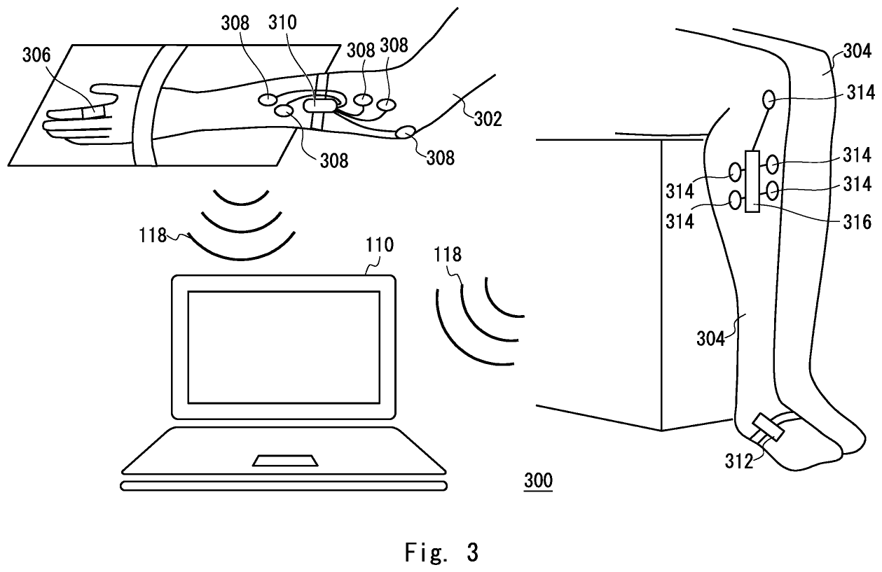

Referring to FIG. 3, an illustration 300 depicts monitoring and determining progress of a patient's rehabilitative treatment in accordance with the present embodiment. More particularly, the illustration 300 depicts physiological performance and range of motion sensing of a portion of a patient's body such as an arm 302 or a leg 304 by the plurality of sensors 108. The plurality of sensors 108 include an IMU device 306 on a finger which measures the movement (i.e., range of motion) of the hand and electrodes 308 of a multichannel EMG device 310 which detects physiological performance of the arm 302 while moving. Similarly, the plurality of sensors 108 include an IMU device 312 on a foot which measures the movement (i.e., range of motion) of the foot and electrodes 314 of a multichannel EMG device 316 which detects physiological performance of the leg 304 while moving. In accordance with the present embodiment, first signals from the EMG devices 310, 316 are transmitted to a processing device 110 such as a laptop and second signals from the IMU devices 306, 312 are transmitted to processing device 110. In this manner, the first and second data are generated in response to the physiological performance and the body portion movement, respectively, for processing by the processing device 110 in accordance with the present embodiment to monitor and determine a patient's progress during rehabilitative treatment.

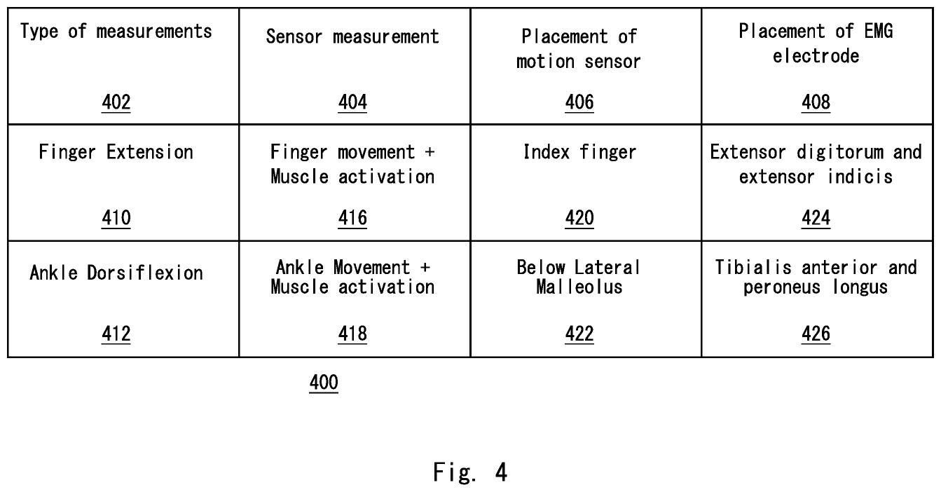

FIG. 4 depicts a table 400 of parameters of the system for monitoring and determining progress of a patient's rehabilitative treatment of FIG. 1 in accordance with the present embodiment. The parameters include types of measurements 402, sensor measurements 404, placement of the motion sensors 406 (e.g., motion sensors 306, 312), placement of EMG electrodes 408 (e.g., EMG electrodes 308, 314). Examples of the type of measurements 402 include a finger extension 410 or ankle dorsiflexion 412.

For the finger extension 410, the sensor measurement would measure finger movement and muscle activation of the finger extension 414. The motion sensor placement 406 would be on, for example, the index finger 420 and the EMG electrode placement would be, for example, on the extensor digitorum muscle location and the extensor indicis muscle location 424.

For the ankle dorsiflexion 412, the sensor measurement would measure muscle activation of the foot 418. The motion sensor placement 406 would be on, for example, the below lateral malleolus 422 and the EMG electrode placement would be, for example, on the tibialis anterior and the peroneus longus 426.

All of these parameters are exemplary and not limiting on the present embodiment. For example, movement of other portions of the body could be measured so long as the motion sensor 406 and EMG electrodes 408 are appropriately placed to sense appropriate physiological performance and body portion movement while the patient 106 moves two or more portions of the patient's body in response to visual and/or auditory instructions. In addition, if the patient 106 is performing rehabilitation sessions at a location different than the clinicians or other professionals 120, the patient 106 will need to be instructed on where to place the sensors for accurate measurements.

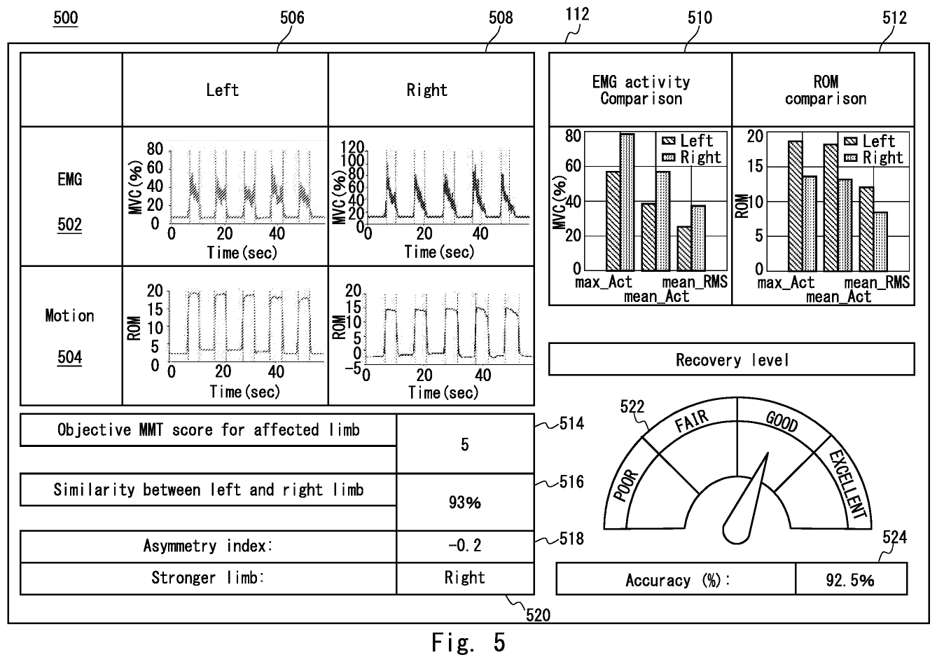

FIG. 5 depicts an illustration 500 of a sample visualization feedback system 112 in accordance with the present embodiment. The visualization feedback system 112 displays EMG signals 502 (i.e., physiological performance signals) and motion signals 504 (i.e., body portion movement signals) for a first portion of the patient's body 506, such as a left lower limb, and a second portion of the patient's body 508, such as a right lower limb. The EMG comparison 510 and range of motion (ROM) comparison 512 are also shown. In accordance with the present embodiment, a calculated objective functional recovery level score 514 is displayed along with the percent similarity between the right and left lower limb 516 an asymmetry index 518 (as will be explained later) and an indication of the stronger limb 520. Finally, a recovery level 522 is displayed (for example in a speedometer read out of poor, fair, good and excellent recovery levels) along with a percent accuracy 524 of the calculation of the recovery level.

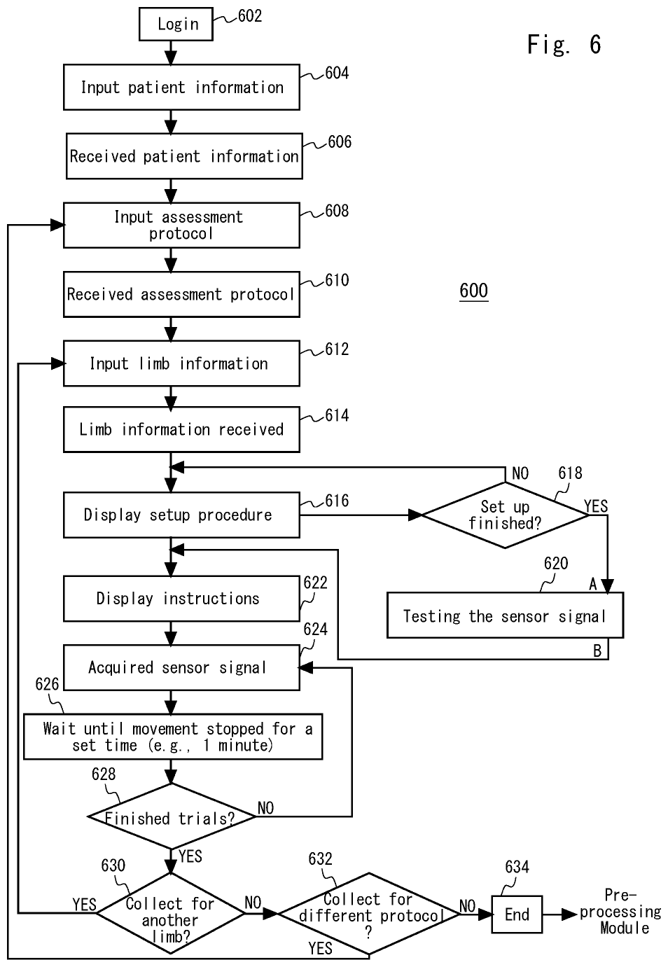

Referring to FIG. 6, a flowchart 600 illustrates a method for patient data acquisition for determining progress of a patient's rehabilitative treatment of the processing device 110 in accordance with the present embodiment. After login at step 602, the patient information is inputted at step 604 such as patient name and demographics and received at step 606. At step 608, the assessment protocol is inputted and at step 610 the assessment protocol is received. Next, at step 612, the first limb information is inputted and at step 614 the second limb information is received.

Next, the display setup procedure is performed at step 616 until it is finished 618. When the display setup procedure is finished 618, the sensor signals are tested 620 and the visual and/or auditory instructions are presented 622 to the patient to perform certain movements of one of the two or more portions of the patient's body (i.e., one of the two limbs). Sensor signals are acquired 624 until movement stops for a predetermined time 626. If the rehabilitation session for that limb is finished 628, data is collected for the other one of the two limbs by returning to input the limb information. If the rehabilitation is not finished, additional sensor signals are acquired 624. When no data is needed to be collected for another limb 630, processing determines at step 632 whether data needs to be collected for a different protocol. If so, processing returns to step 608 to input the different assessment protocol. If not, data collection ends and the first data generated in response to the sensed movement of the first limb and the second data generated in response to the sensed movement of the second limb are forwarded to the preprocessing module 122 of the analytic engine 114 (FIG. 1).

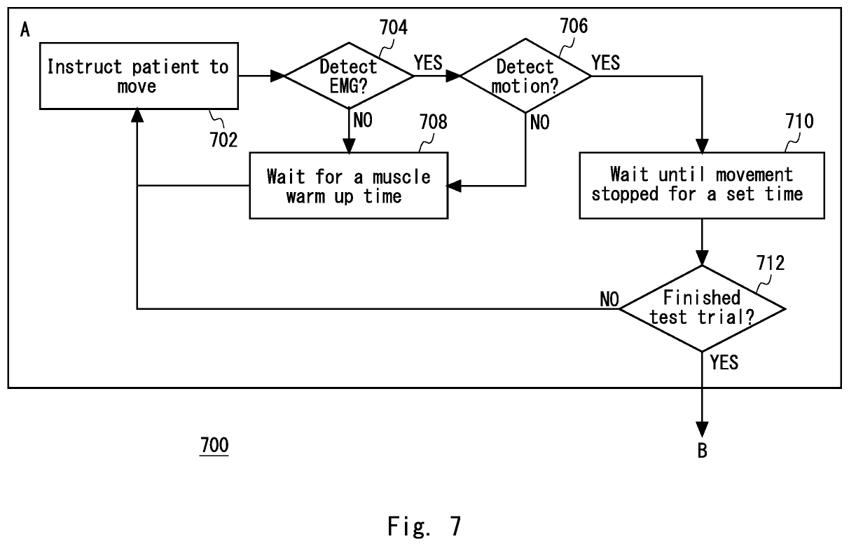

FIG. 7 depicts a flowchart 700 illustrating the method for testing the sensor signals 620. First, the patient is instructed to move at step 702. If either the EMG signal 704 or the motion 706 is not detected, processing waits for a muscle warm up time at step 708 before again instructing the patient to move at step 702. When both the EMG signal 704 and the motion 706 is detected, movement is stopped for a predetermined time 710 before the sensor signal test trial is continued 712. When the sensor signal test trial is finished 712 processing returns to step 622 (FIG. 6).

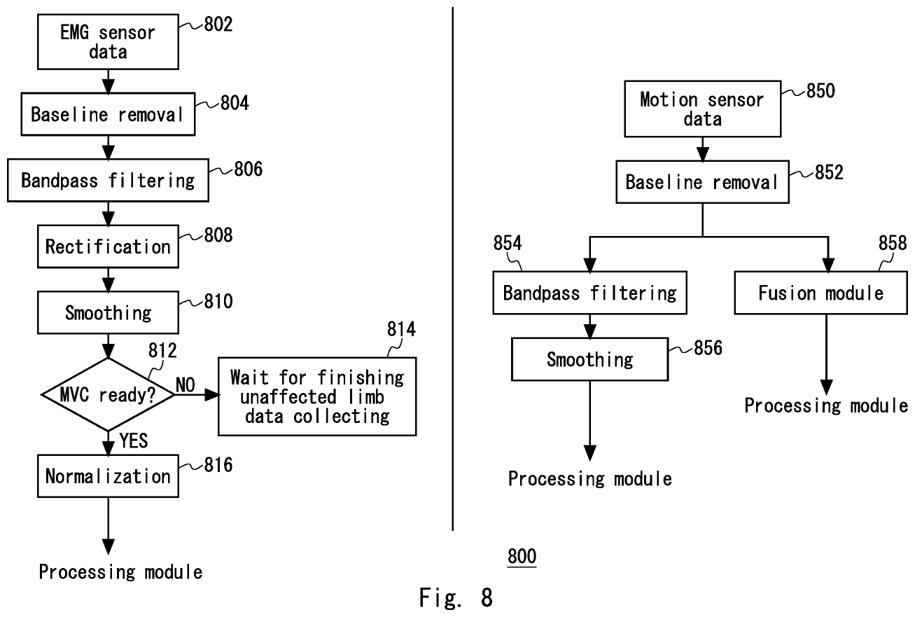

FIG. 8 depicts a flowchart 800 illustrating sensor signal preprocessing in accordance with the present embodiment. For the EMG signal 802, pre-processing involves baseline removal 804, bandpass filtering 806, signal rectification 808 and signal smoothing 810. If sufficient unaffected limb data has not been received 812, processing waits for the unaffected limb data collection to finish 814. When the EMG maximum volume contraction (MVC) data is ready 812 the signal is normalized 816 and passed to the processing module.

For the motion sensor signal 850, pre-processing involves baseline removal 852. Then the signal is bandpass filtered 854 and smoothed 856 before being passed to the processing module. The signal is also passed to a fusion module 858 after which it is passed to the processing module.

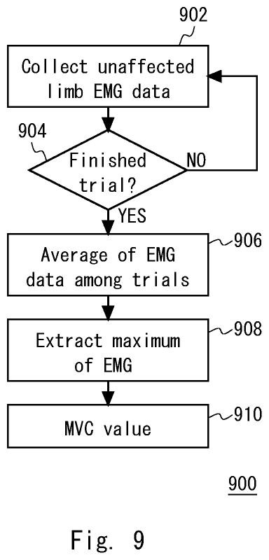

FIG. 9 depicts a flowchart 900 illustrating maximum volume contraction (MVC) of electromyography (EMG) extraction in accordance with the present embodiment. Unaffected limb data is collected 902 until the trial is finished 904. When the trial is finished 904, the average of the EMG data collected during the trial is calculated 906. The maximum of averaged EMG is then extracted 908 and used as the MVC value 910.

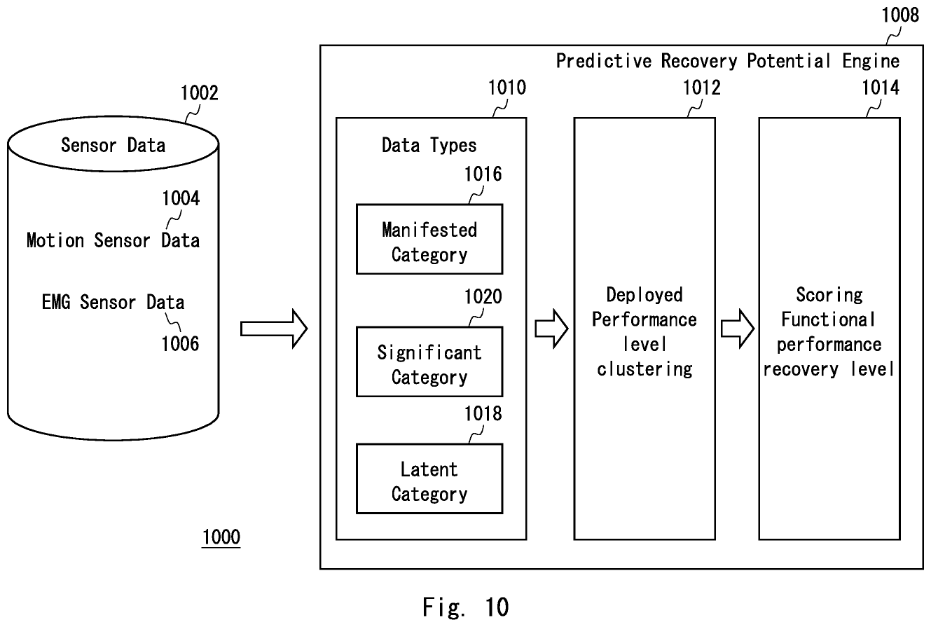

Referring to FIG. 10, a block diagram 1000 depicts processing portions of objective scoring module 124 (FIG. 1) in accordance with the present embodiment. Sensor data 1002 collects and stores IMU sensor data 1004 and EMG sensor data 1006 for later processing. In this manner, the patient's data can be obtained as first and second signals from two or more sensing devices while a portion of the patient's body (e.g., upper limb or lower limb) is moved and can be stored for later processing by a predictive recovery potential engine 1008. The predictive recovery engine 1008 includes a scoring module 510, a deployed performance level clustering module 1012 and a scoring functional performance recovery level module 1014. The scoring module 1010 determines the manifested category 1016 in response to the second signal (i.e., the motion sensor data), determines a latent category 1018 in response to the second signal (i.e., the EMG sensor data), and determines the significant category 1020 in response to both the first signal and the second signal. By applying all three categories (manifested, latent and significant) in accordance with the present embodiment, the prediction of potential recovery of patients and planning of rehabilitative treatment strategies can be optimized.

The deployed performance level clustering module 1012 determines a plurality of classifiers from the latent category 1018, the manifested category 1016 and the significant category 1020. The scoring functional performance recovery level module 1014 determines an objective score representing the patient's rehabilitative treatment progress in response to the plurality of classifiers of the latent category 1018, the manifested category 1016 and the significant category 1020.

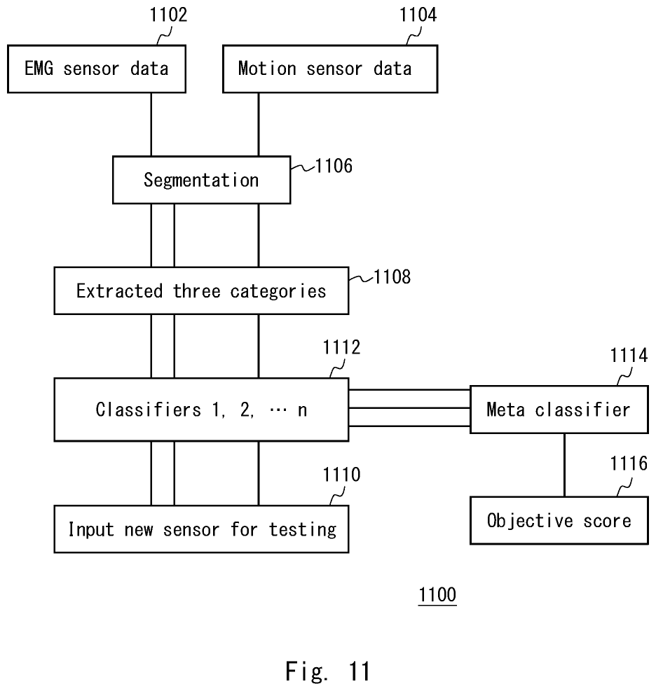

FIG. 11 depicts a block diagram 1100 of the objective score module 124 in accordance with the present embodiment. The EMG sensor data 1102 and the motion sensor data 1104 are segmented 1106 to generate the first and second data for the two limbs. The manifested category, a latent category and significant category of the first data and the second data are extracted 1108. The extracted three categories 1108 and new sensor data 1110 are processed to identify n number of classifiers for scoring functional recovery 1112. The classifiers are combined to generate a meta classifier 1114 form which the objective functional recovery level is calculated 1116.

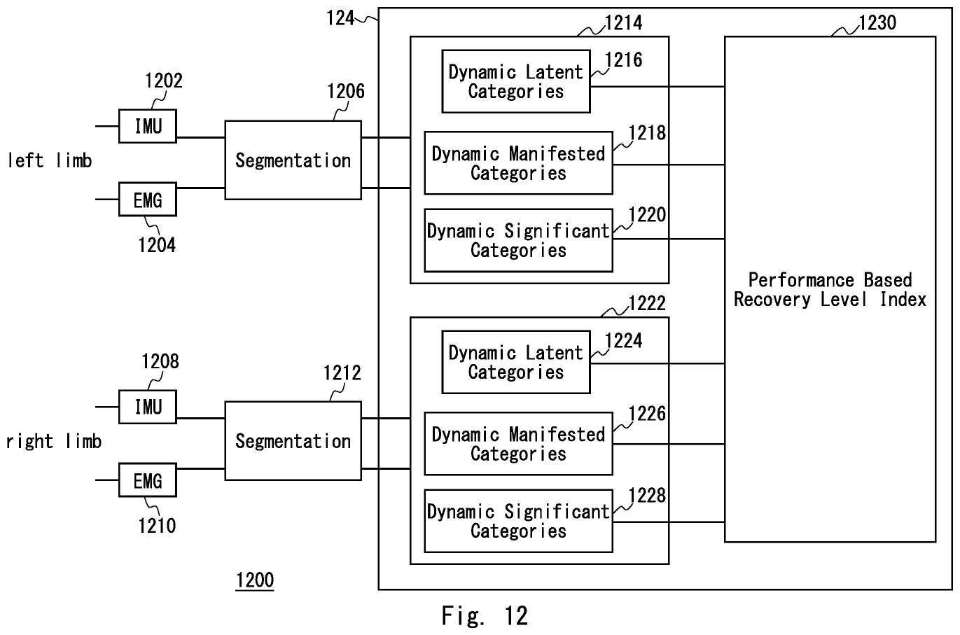

FIG. 12 depicts a block diagram 1200 of recovery level index determination of the objective scoring module 124 (FIG. 1) in accordance with the present embodiment. The motion sensing signal 1202 and the EMG sensing signal 1204 are segmented 1206 to generate the first data from a left limb. The motion sensing signal 1208 and the EMG sensing signal 1210 are segmented 1212 to generate the second data from a right limb. The objective scoring module 124 has a left limb scoring module 1214 and a right limb scoring module. The left limb scoring module 1214 receives the first data categorizes dynamic latent categories 1216, dynamic manifested categories 1218, and dynamic significant categories 1220 within the first data. The right limb scoring module 1222 receives the first data categorizes dynamic latent categories 1224, dynamic manifested categories 1226, and dynamic significant categories 1228 within the second data. All of the latent category 1216, 1224, the manifested category 1218, 1226 and the significant category 1220, 1228 of the first data and the second data are used to determine the objective performance-based recovery level index 1230 representing the patient's rehabilitative treatment progress.

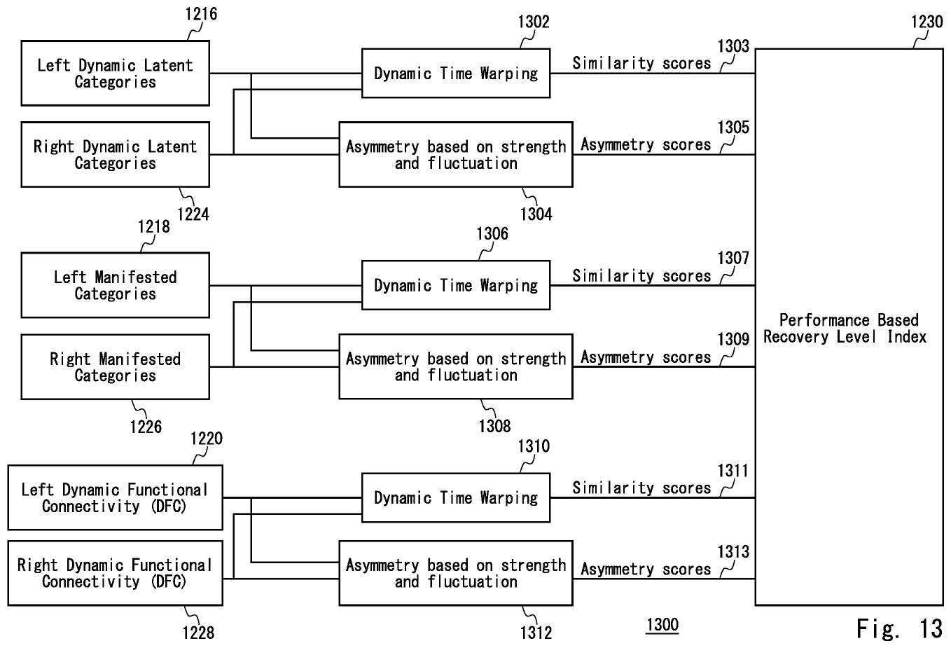

FIG. 13 depicts a more detailed block diagram of post-segmentation recovery level index determination by the objective scoring module 124 in accordance with the present embodiment. The left and right dynamic latent categories 1216, 1224 are processed by dynamic time warping 1302 to generate a similarity score 1303 for calculating the performance based recovery level 1230. In addition, the left and right dynamic latent categories 1216, 1224 are processed by strength and fluctuation based asymmetry 1304 to generate an asymmetry score 1305 for calculating the performance based recovery level 1230.

In a similar manner, the left and right dynamic manifested categories 1218, 1226 are processed by dynamic time warping 1306 to generate a similarity score 1307 for calculating the performance based recovery level 1230 and the left and right dynamic manifested categories 1218, 1226 are processed by strength and fluctuation based asymmetry 1308 to generate an asymmetry score 1309 for calculating the performance based recovery level 1230.

Finally, the left and right dynamic functional connectivity categories (i.e., dynamic significant categories) 1220, 1228 are processed by dynamic time warping 1310 to generate a similarity score 1311 for calculating the performance based recovery level 1230. In addition, the left and right dynamic functional connectivity categories 1220, 1228 are processed by strength and fluctuation based asymmetry 1312 to generate an asymmetry score 1313 for calculating the performance based recovery level 1230.

In this manner, the asymmetry index 518 and the similarity percentage 516 (FIG. 5) can also be calculated from the similarity scores 1303, 1307, 1311 and the asymmetry scores 1305, 1309, 1312, respectively.

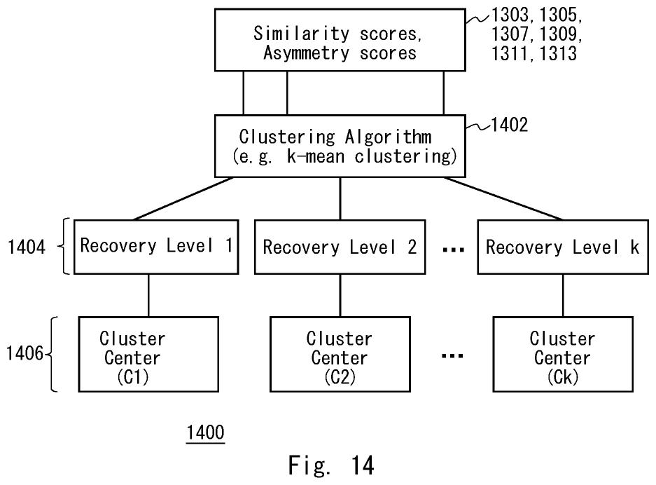

Referring to FIG. 14, a flowchart 1400 depicts clustering in accordance with the present embodiment to calculate the recovery level 522 (FIG. 5). The similarity scores 1303, 1307, 1311 and the asymmetry scores 1305, 1309, 1313 are clustered 1402 in accordance with a clustering algorithm such as a k-mean clustering algorithm. Using the clusters defined by the clustering algorithm 1402, the recovery levels 1404 are defined by a centre of each cluster 1406.

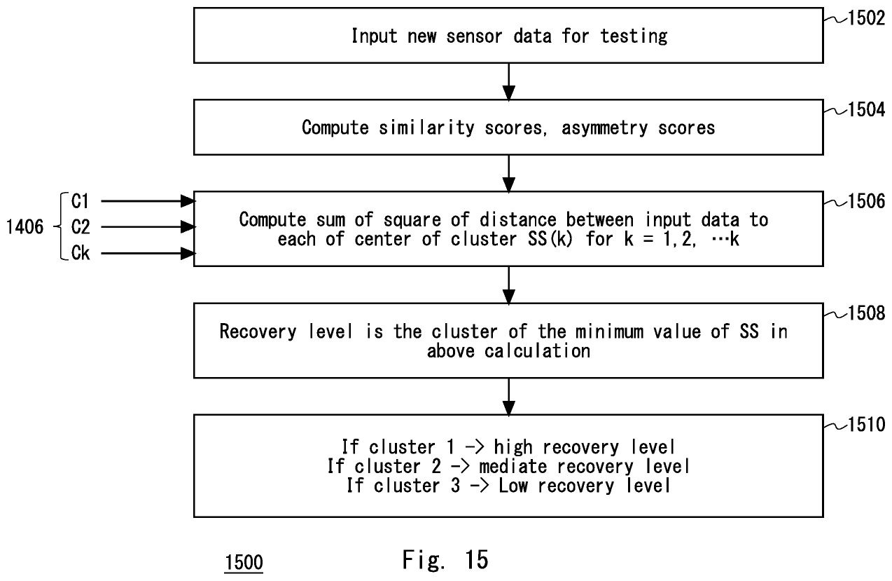

Referring to FIG. 15, depicts a flowchart 1500 for determining recovery level in response to the clustering in accordance with the flowchart 1400 in accordance with the present embodiment is depicted. When new sensor data is received 1502, the similarity scores and asymmetry scores are calculated 1504. Using the cluster centres 1406, a sum of the square of the distance between the inputs data to each cluster centre is calculated 1506. The recovery level is determined from the cluster of the minimum values of the sum of the square of the distances 1508 and the output of recovery level 522 (FIG. 5) is determined from the cluster 1510.

Thus, it can be seen that the present embodiment provides an objective assessment of motor function and quantification of functional abilities which reflects the individual patient's functional abilities whether obtained in person in early stage recovery or obtained later by telemonitoring a patient's recovery. In accordance with the present embodiment, a system and method is provided to determine the objective functional recovery level representing the patient's rehabilitative treatment progress in response to corresponding segmented portions of the first data and the second data taken from respective first and second limbs of the patient.

In the present disclosure, arbitrary processing can be also implemented by causing a CPU (Central Processing Unit) to execute a computer program. The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

While exemplary embodiments have been presented in the foregoing detailed description of the invention, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing an exemplary embodiment of the invention, it being understood that various changes may be made in the function and arrangement of steps and method of operation described in the exemplary embodiment without departing from the scope of the invention as set forth in the appended claims.

For example, the whole or part of the exemplary embodiments disclosed above can be described as, but not limited to, the following supplementary notes.

### (Supplementary Note 1)

A method for monitoring and determining progress of a patient's rehabilitative treatment, the method comprising:

sensing physiological performance and body portion movement while moving two or more portions of the patient's body in response to visual and/or auditory instructions;

generating first data in response to the sensed movement of a first portion of the patient's body;

generating second data in response to the sensed movement of a second portion of the patient's body; and

determining an objective functional recovery level representing the patient's rehabilitative treatment progress in response to all of a manifested category, a latent category and a significant category of the first data and the second data.

### (Supplementary Note 2)

The method in accordance with Supplementary note 1 wherein generating the first data comprises:

generating first physiological performance data in response to the sensed physiological performance of the first portion of the patient's body;

generating first sensed movement data in response to the sensed movement of the first portion of the patient's body; and

segmenting the first physiological performance data and the first sensed movement data to generate the first data.

### (Supplementary Note 3)

The method in accordance with Supplementary note 2 wherein generating the second data comprises:

generating second physiological performance data in response to the sensed physiological performance of the second portion of the patient's body;

generating second sensed movement data in response to the sensed movement of the second portion of the patient's body; and

segmenting the second physiological performance data and the second sensed movement data to generate the second data.

### (Supplementary Note 4)

The method in accordance with Supplementary note 3 wherein determining the objective functional recovery level comprises determining the objective functional recovery level representing the patient's rehabilitative treatment progress in response to corresponding segmented portions of the first data and the second data.

### (Supplementary Note 5)

The method in accordance with any of Supplementary notes 1 to 4 wherein the first portion of the patient's body is an affected portion of the patient's body requiring rehabilitative treatment and wherein the second portion of the patient's body is an unaffected portion of the patient's body which is healthy.

### (Supplementary Note 6)

The method in accordance with any of Supplementary notes 1 to 5 wherein determining an objective functional recovery level comprises:

transmitting the first data and the second data from a first location where the patient is responding to the visual and/or auditory instructions to a second location; and

processing the first and second data at the second location to determine the objective functional recovery level representing the patient's rehabilitative treatment progress.

### (Supplementary Note 7)

The method in accordance with Supplementary note 2 wherein generating the first physiological performance data comprises generating the first physiological performance data in response to an electromyography (EMG) of the sensed physiological performance of the first portion of the patient's body, and wherein generating the first sensed movement data comprises generating the first sensed movement data in response to inertial measurement units (IMU) of the sensed movement of the first portion of the patient's body.

### (Supplementary Note 8)

The method in accordance with Supplementary note 3 wherein generating the second physiological performance data comprises generating the second physiological performance data in response to an electromyography (EMG) of the sensed physiological performance of the second portion of the patient's body, and wherein generating the second sensed movement data comprises generating the second sensed movement data in response to inertial measurement units (IMU) of the sensed movement of the second portion of the patient's body.

### (Supplementary Note 9)

The method in accordance with Supplementary note 5 wherein the first portion of the patient's body moved comprises an affected one of the patient's arm, the patient's leg, the patient's hand, the patient's foot, the patient's fingers or the patient's toes, and wherein the second portion of the patient's body moved comprises an unaffected other one of the patient's arm, the patient's leg, the patient's hand, the patient's foot, the patient's fingers or the patient's toes.

### (Supplementary Note 10)

A system for monitoring and determining progress of a patient's rehabilitative treatment, the system comprising:

a patient output device for displaying visual and/or presenting auditory instructions to the patient;

a plurality of sensory devices for sensing physiological performance and body portion movement while the patient moves two or more portions of the patient's body in response to the visual and/or auditory instructions; and

a processing means for generating first data in response to the sensed movement of a first portion of the patient's body, generating second data in response to the sensed movement of a second portion of the patient's body, and determining an objective functional recovery level representing the patient's rehabilitative treatment progress in response to all of a manifested category, a latent category and a significant category of the first data and the second data.

### (Supplementary Note 11)

The system in accordance with Supplementary note 10 wherein the processing means generates the first data by generating first physiological performance data in response to the sensed physiological performance of the first portion of the patient's body, generating first sensed movement data in response to the sensed movement of the first portion of the patient's body, and segmenting the first physiological performance data and the first sensed movement data to generate the first data.

### (Supplementary Note 12)

The system in accordance with Supplementary note 11 wherein the processing means generates the second data by generating second physiological performance data in response to the sensed physiological performance of the second portion of the patient's body, generating second sensed movement data in response to the sensed movement of the second portion of the patient's body, and segmenting the second physiological performance data and the second sensed movement data to generate the second data.

### (Supplementary Note 13)

The system in accordance with Supplementary note 12 wherein the processing means determines the objective functional recovery level by determining the objective functional recovery level representing the patient's rehabilitative treatment progress in response to corresponding segmented portions of the first data and the second data.

### (Supplementary Note 14)

The system in accordance with any of Supplementary notes 10 to 13 wherein the first portion of the patient's body is an affected portion of the patient's body requiring rehabilitative treatment and wherein the second portion of the patient's body is an unaffected portion of the patient's body which is healthy.

### (Supplementary Note 15)

The system in accordance with any of Supplementary notes 10 to 14 wherein the processing means comprises:

first processing means for generating the first data in response to the sensed movement of the first portion of the patient's body and generating the second data in response to the sensed movement of the second portion of the patient's body; and

second processing means for determining the objective functional recovery level representing the patient's rehabilitative treatment progress in response to all of the manifested category, the latent category and the significant category of the first data and the second data,

the system further comprising transceiving means coupled between the first processing means and the second processing means, the transceiving means transmitting the first data and the second data from a first location where the patient is responding to the visual and/or auditory instructions to a second location wherein the objective functional recovery level representing the patient's rehabilitative treatment progress is determined.

### (Supplementary Note 16)

The system in accordance with Supplementary note 11 wherein the processing means generates the first physiological performance data in response to an electromyography (EMG) of the sensed physiological performance of the first portion of the patient's body and generates the first sensed movement data in response to inertial measurement units (IMU) of the sensed movement of the first portion of the patient's body.

### (Supplementary Note 17)

The system in accordance with Supplementary note 12 wherein the processing means generates the second physiological performance data in response to an electromyography (EMG) of the sensed physiological performance of the second portion of the patient's body and generates the second sensed movement data in response to inertial measurement units (IMU) of the sensed movement of the second portion of the patient's body.

### (Supplementary Note 18)

The system in accordance with Supplementary note 14 wherein the first portion of the patient's body moved comprises an affected one of the patient's arm, the patient's leg, the patient's hand, the patient's foot, the patient's fingers or the patient's toes, and wherein the second portion of the patient's body moved comprises an unaffected other one of the patient's arm, the patient's leg, the patient's hand, the patient's foot, the patient's fingers or the patient's toes.

### (Supplementary Note 19)

A non-transitory computer readable medium containing program instructions for causing a computer to perform a method for monitoring and determining progress of a patient's rehabilitative treatment comprising:

receiving first physiological performance data in response to a sensed physiological performance of a first portion of the patient's body;

receiving first sensed movement data in response to a sensed movement of the first portion of the patient's body;

segmenting the first physiological performance data and the first sensed movement data to generate first data;

receiving second physiological performance data in response to a sensed physiological performance of a second portion of the patient's body;

receiving second sensed movement data in response to a sensed movement of the second portion of the patient's body;

segmenting the second physiological performance data and the second sensed movement data to generate second data; and

determining an objective functional recovery level representing the patient's rehabilitative treatment progress in response to all of a manifested category, a latent category and a significant category of corresponding segmented portions of the first data and the second data.

This application is based upon and claims the benefit of priority from Singapore Patent Application No. 10201800971R, filed on Feb. 5, 2018, the disclosure of which is incorporated herein in its entirety by reference.

### REFERENCE SIGNS LIST

102 PATIENT OUTPUT DEVICE
104 DISPLAY
108 SENSOR DEVICE
110 PROCESSING DEVICE
112 USER INTERFACE
114 ANALYTIC ENGINE
306, 312 IMU DEVICE

What is claimed is:

1. A method for monitoring and determining progress of a patient's rehabilitative treatment, the method comprising:
sensing physiological performance and body portion movement while moving two or more portions of the patient's body in response to visual and/or auditory instructions;
generating first data in response to the sensed movement of a first portion of the patient's body;
generating second data in response to the sensed movement of a second portion of the patient's body; and
determining an objective functional recovery level representing the patient's rehabilitative treatment progress in response to all of a manifested category, a latent category and a significant category of the first data and the second data.

2. The method in accordance with claim 1 wherein generating the first data comprises:
generating first physiological performance data in response to the sensed physiological performance of the first portion of the patient's body;
generating first sensed movement data in response to the sensed movement of the first portion of the patient's body; and
segmenting the first physiological performance data and the first sensed movement data to generate the first data.

3. The method in accordance with claim 2 wherein generating the second data comprises:
generating second physiological performance data in response to the sensed physiological performance of the second portion of the patient's body;
generating second sensed movement data in response to the sensed movement of the second portion of the patient's body; and
segmenting the second physiological performance data and the second sensed movement data to generate the second data.

4. The method in accordance with claim 3 wherein determining the objective functional recovery level comprises determining the objective functional recovery level representing the patient's rehabilitative treatment progress in response to corresponding segmented portions of the first data and the second data.

5. The method in accordance with claim 1 wherein the first portion of the patient's body is an affected portion of the patient's body requiring rehabilitative treatment and wherein the second portion of the patient's body is an unaffected portion of the patient's body which is healthy.

6. The method in accordance with claim 1 wherein determining an objective functional recovery level comprises:
transmitting the first data and the second data from a first location where the patient is responding to the visual and/or auditory instructions to a second location; and
processing the first and second data at the second location to determine the objective functional recovery level representing the patient's rehabilitative treatment progress.

7. The method in accordance with claim 2 wherein generating the first physiological performance data comprises generating the first physiological performance data in response to an electromyography (EMG) of the sensed physiological performance of the first portion of the patient's body, and wherein generating the first sensed movement data comprises generating the first sensed movement data in response to inertial measurement units (IMU) of the sensed movement of the first portion of the patient's body.

8. The method in accordance with claim 3 wherein generating the second physiological performance data comprises generating the second physiological performance data in response to an electromyography (EMG) of the sensed physiological performance of the second portion of the patient's body, and wherein generating the second sensed movement data comprises generating the second sensed movement data in response to inertial measurement units (IMU) of the sensed movement of the second portion of the patient's body.

9. The method in accordance with claim 5 wherein the first portion of the patient's body moved comprises an affected one of the patient's arm, the patient's leg, the patient's hand, the patient's foot, the patient's fingers or the patient's toes, and wherein the second portion of the patient's body moved comprises an unaffected other one of the patient's arm, the patient's leg, the patient's hand, the patient's foot, the patient's fingers or the patient's toes.

10. A system for monitoring and determining progress of a patient's rehabilitative treatment, the system comprising:
a patient output device configured to display visual and/or presenting auditory instructions to the patient;
a plurality of sensory devices configured to sense physiological performance and body portion movement while the patient moves two or more portions of the patient's body in response to the visual and/or auditory instructions; and
a processing device, the processing device comprising at least one memory storing computer program and at least one processor configured to execute the computer program to:
generate first data in response to the sensed movement of a first portion of the patient's body, generate second data in response to the sensed movement of a second portion of the patient's body, and determine an objective functional recovery level representing the patient's rehabilitative treatment progress in response to all of a manifested category, a latent category and a significant category of the first data and the second data.

11. The system in accordance with claim 10 wherein the at least one processor is further configured to execute the computer program to generate the first data by generating first physiological performance data in response to the sensed physiological performance of the first portion of the patient's body, generating first sensed movement data in response to the sensed movement of the first portion of the patient's body, and segmenting the first physiological performance data and the first sensed movement data to generate the first data.

12. The system in accordance with claim 11 wherein the at least one processor is further configured to execute the computer program to generate the second data by generating second physiological performance data in response to the sensed physiological performance of the second portion of the patient's body, generating second sensed movement data in response to the sensed movement of the second portion of the patient's body, and segmenting the second physiological performance data and the second sensed movement data to generate the second data.

13. The system in accordance with claim 12 wherein the at least one processor is further configured to execute the computer program to determine the objective functional recovery level by determining the objective functional recovery level representing the patient's rehabilitative treatment progress in response to corresponding segmented portions of the first data and the second data.

14. The system in accordance with claim 10 wherein the first portion of the patient's body is an affected portion of the patient's body requiring rehabilitative treatment and wherein the second portion of the patient's body is an unaffected portion of the patient's body which is healthy.

15. The system in accordance with claim 10 wherein the processing device comprises a first processing device and a second processing device, the first processing device comprising at least one first memory storing first computer program and at least one first processor configured to execute the first computer program to generate the first data in response to the sensed movement of the first portion of the patient's body and generate the second data in response to the sensed movement of the second portion of the patient's body; and the second processing device comprising at least one second memory storing second computer program and at least one second processor configured to execute the second computer program to determine the objective functional recovery level representing the patient's rehabilitative treatment progress in response to all of the manifested category, the latent category and the significant category of the first data and the second data,
the system further comprising transceiver coupled between the first processing device and the second processing device, the transceiver transmitting the first data and the second data from a first location where the patient is responding to the visual and/or auditory instructions to a second location wherein the objective functional recovery level representing the patient's rehabilitative treatment progress is determined.

16. The system in accordance with claim 11 wherein the at least one processor is further configured to execute the computer program to generate the first physiological performance data in response to an electromyography (EMG) of the sensed physiological performance of the first portion of the patient's body and generate the first sensed movement data in response to inertial measurement units (IMU) of the sensed movement of the first portion of the patient's body.

17. The system in accordance with claim 12 wherein the at least one processor is further configured to execute the computer program to generate the second physiological performance data in response to an electromyography (EMG) of the sensed physiological performance of the second portion of the patient's body and generate the second sensed movement data in response to inertial measurement units (IMU) of the sensed movement of the second portion of the patient's body.

18. The system in accordance with claim 14 wherein the first portion of the patient's body moved comprises an affected one of the patient's arm, the patient's leg, the patient's hand, the patient's foot, the patient's fingers or the patient's toes, and wherein the second portion of the patient's body moved comprises an unaffected other one of the patient's arm, the patient's leg, the patient's hand, the patient's foot, the patient's fingers or the patient's toes.

19. A non-transitory computer readable medium containing program instructions for causing a computer to perform a method for monitoring and determining progress of a patient's rehabilitative treatment comprising:
receiving first physiological performance data in response to a sensed physiological performance of a first portion of the patient's body;
receiving first sensed movement data in response to a sensed movement of the first portion of the patient's body;
segmenting the first physiological performance data and the first sensed movement data to generate first data;
receiving second physiological performance data in response to a sensed physiological performance of a second portion of the patient's body;
receiving second sensed movement data in response to a sensed movement of the second portion of the patient's body;
segmenting the second physiological performance data and the second sensed movement data to generate second data; and
determining an objective functional recovery level representing the patient's rehabilitative treatment progress in response to all of a manifested category, a latent category and a significant category of corresponding segmented portions of the first data and the second data.
